(19) United States
(12) Patent Application Publication
(10) Pub. No.: US 2009/0208918 A1
(43) Pub. Date: Aug. 20, 2009

(54) METHODS AND DEVICES FOR EX-VIVO MAINTENANCE OF BONE MARROW, HEMATOPOIESIS AND BLOOD CELL PRODUCTION

(76) Inventor: Daniel Kraft, Stanford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/370,541

(22) Filed: Feb. 12, 2009

Related U.S. Application Data
(60) Provisional application No. 61/064,055, filed on Feb. 13, 2008.

(51) Int. Cl. A01N 1/02; C12N 5/00; C12N 5/08; C12Q 1/02

(52) U.S. Cl. 435/1.2; 435/374; 435/372; 435/284.1; 435/29

(57) ABSTRACT

Devices and methods are provided for the ex vivo maintenance of bone marrow ‘niches’ or normal physiologic home of bone marrow (BM). Intact bone marrow is perfused with a medium that supports cell viability. Stem and progenitor cells may be maintained, expanded or differentiated within the system, screened for responses to various agents, or used to generate blood products.

### BACKGROUND OF THE INVENTION

Bone marrow is a complex, three dimensional structure, filled with proliferating and differentiating cells within connective stromal and endosteal tissue, bordered by venous sinuses and microvasculature, and is the ‘home/niche’ or ‘factory’ for blood forming stem cells and mature blood cell production. Several distinct stem cells are present in bone marrow, including mesenchymal stem cells and hematopoietic stem cells. Hematopoietic Stem Cells (HSC) are the most extensively characterized adult stem cell, yet understanding of their human niche, and the ability to maintain and expand HSCs in vitro remains a challenge.

Blood/marrow/hematopoietic stem cell harvest, transplantation, homing and engraftment all occurs within the context of the marrow hematopoietic niche. The importance and role of the marrow niche is evident in the difficulty to date in the ability to successfully expand hematopoietic stem cells (HSC) ex vivo. A deeper understanding of mammalian hematopoiesis, the hematopoietic stem cell niche, and its normal and diseased state, could lead to improved outcomes in clinical bone marrow transplantation, through improved methods to condition the niche for transplant and to mobilize, and directly harvest stem cells from their endogenous niche, as well as the ability to optimize transplant/homing/engraftment of donor stem cells. Better understanding and means to study the niche in the context of hematologic diseases (i.e. myelodysplastic disease, leukemias, thalasemias) and malignancies (i.e. leukemias, myeloma) may also enable improved therapies for marrow and HSC derived diseases.

Understanding of human hematopoiesis is hampered by lack of effective model systems in which to study the human blood forming organ. In vitro study of the HSC niche has generally been limited to well-based cultures, or use of 3 dimensional structures, supplemented with cytokines. Xenotransplantion models of human CD34⁺ cells in immunodeficient mice provide for generally low levels of human engraftment, and in the context of a murine marrow environment. Other models, such as SCID-hu mice with transplanted intact human marrow, are laborious to create and limited by available fetal donor tissues. Clinical and ethical issues make it difficult to study human marrow in living patients.

The interaction of stem cells with their microenvironment provides important cues for their maintenance, proliferation and differentiation. This physical environment in which stem cells reside may be referred to as the stem cell microenvironment, or niche. The stromal and other cells involved in this niche provide soluble and bound factors, which have a multitude of effects. For example, two protein families, the TGFβ family and Wnt family, are known to be involved in stem cell regulation, and have been found to be operative in a number of systems and different organisms.

Cell-cell and cell to extracellular matrix (ECM) interactions are important for both the induction and maintenance of differentiation in several cell lineages. In the case of the ECM, specific cell surface receptors can bind to particular components of the ECM, activating an intracellular signal transduction pathway that is analogous to the signaling pathways that have been identified for polypeptide growth factors and growth inhibitors. The extracellular matrix may also sequester and modulate the local concentration of secreted factors available within the stem cell niche.

Other signals that control stem cells require direct cell-cell contact. For example, interaction between the receptor Notch and its ligand Delta, both of which are transmembrane molecules, requires cell to cell contact. Notch signaling is involved in regulation of embryonic and adult tissues of vertebrates; examples include retinal neuroepithelium, skeletal muscle, and blood.

Various models have been proposed for the interaction between stem cell and niche. Methods that allow for efficient in vitro maintenance of hematopoietic niches are of interest for therapeutic and research purposes. The present invention addresses this issue.

### SUMMARY OF THE INVENTION

Devices and methods are provided for the ex-vivo maintenance of bone marrow ‘niches’ or the normal physiologic home of bone marrow (BM). In the methods of the invention, intact bone marrow, which optionally includes bone normally associated with the marrow, and/or additional protective coatings, is perfused with a medium that supports bone marrow cell viability in an in vitro culture system. The perfused medium may further comprise biologically active agents, including, without limitation, factors such as cytokines, drug candidates, cells, viruses, polynucleotides, and the like. The cells present (and in some cases their offspring) in the bone marrow maintain viability and various aspects of their biological function for a period of time ranging from at least about one day, at least about 3 days, at least about one week, at least about two weeks, at least about 3 weeks, or longer.

In some embodiments of the invention, the bone marrow is human bone marrow, in other embodiments the bone marrow is obtained from a non-human mammal, e.g. bovine, porcine, etc. In some embodiments, for example where the bone marrow if human, the bone marrow is contained as a “core” comprising the cancellous bone and non-bony cells of the marrow, which are maintained in the original three-dimensional structure found in vivo. Such “cores” are optionally coated with a protective layer, e.g. a wax or other coating. In other embodiments the bone marrow includes bone normally contacting the marrow in vivo. Bones of interest include, without limitation, cross-section of long bones, vertebrae, and the like.

Perfusion of the bone marrow is important to the function, and the systems of the invention include such elements that are required to maintain perfusion, which may include a pump, optionally in combination with controllers, filters, media reservoir, etc., and such vessels as are required to provide for perfusion, e.g. tubing, etc. The outer surface of the bone or bone marrow may or may not be in contact with medium. In some embodiments, the outer surface of the bone or bone marrow in contact with a vessel, e.g. a sterile vessel, which optionally includes a matrix to support the bone, e.g. collagen, matrigel, etc.

Perfusion of the marrow may provide for multiple passively perfused channels; syringe pump via single or multiple proximal/distal entry site(s); cultured core biopsies, perfused cores; perfusion through the endogenous vasculature, and the like.

In some embodiments of the invention, hematopoiesis, including formation of new red blood cells, white blood cells and platelets, is maintained in the system. In such embodiments, the ex vivo hematopoietic niche, which may be referred to herein as a ‘Bone Marrow Bioreactor’ (BMB) or Bone Marrow Unit (BMU), may be maintained and utilized for production of blood products such as new blood cells, including red blood cells (RBC), white blood cells (WBC: leukocytes, B-cells, T-cells, neutrophils and granulocytes, platelets, etc., which cells may be collected or removed from the system, and are optionally utilized for therapeutic purposes, e.g. in the transfusion of patients requiring red blood cells, platelets, neutrophils, progenitor cells, etc., for research purposes, and the like. The ability to expand existing hematopoietic stem cells (HSC), and hematopoietic progenitor cells within such a ‘bone marrow niche bioreactor’ (with or without prior ‘conditioning’ of the BMU) and/or the ability to place donor hematopoietic or other stem cell populations within the BMB/BMU provides a means to maintain and/or expand their numbers of these stem cells for use in research or clinical settings.

In other embodiments of the invention, the bone marrow bioreactor is utilized in drug screening. Screening methods may involve contacting all or a portion of the bone marrow bioreactor with a candidate drug, where the effect of the drug on viability, function, etc., of the bone marrow niche or cells involved therewith is determined. Screening may include determining the effect of a candidate drug screening of drugs and any or all molecules which may affect various aspects, and function of the stromal and other cellular and structural components of the hematopoietic BM niche, of a mesenchymal stem cell niche, or the blood forming stem and progenitor cells which reside in bone marrow. Screening, testing and development of existing or novel drugs and compounds may be tested on normal hematopoiesis, as well as on malignant cells (i.e. leukemia, lymphoma, myeloma cells, solid tumor metastasis and various cancer stem cells) which can reside within marrow.

### BRIEF DESCRIPTION OF THE DRAWINGS

FIG. 1. Bone Marrow and the hematopoietic niche: Illustrative drawings of bone marrow, residing with bone marrow cavity of a long bone, the various cells and components.

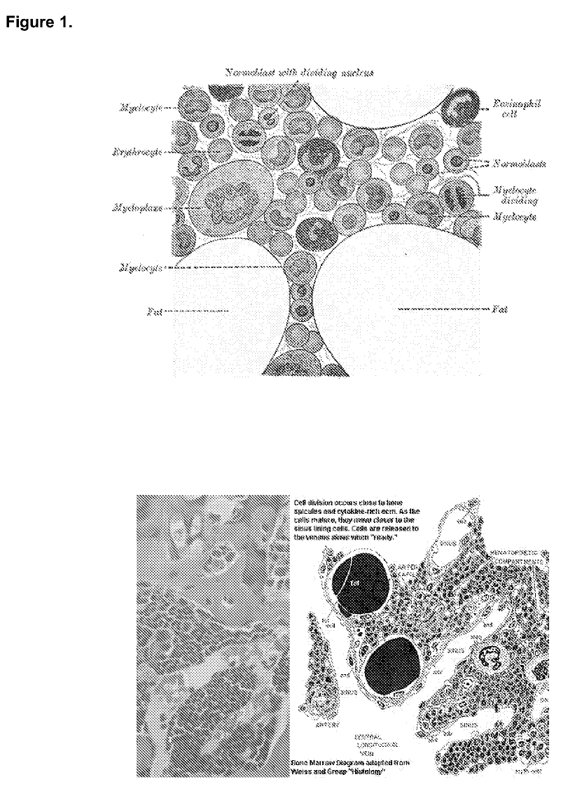

FIG. 2. Individual marrow filled vertebrae have isolatable arterial and venous blood supply.

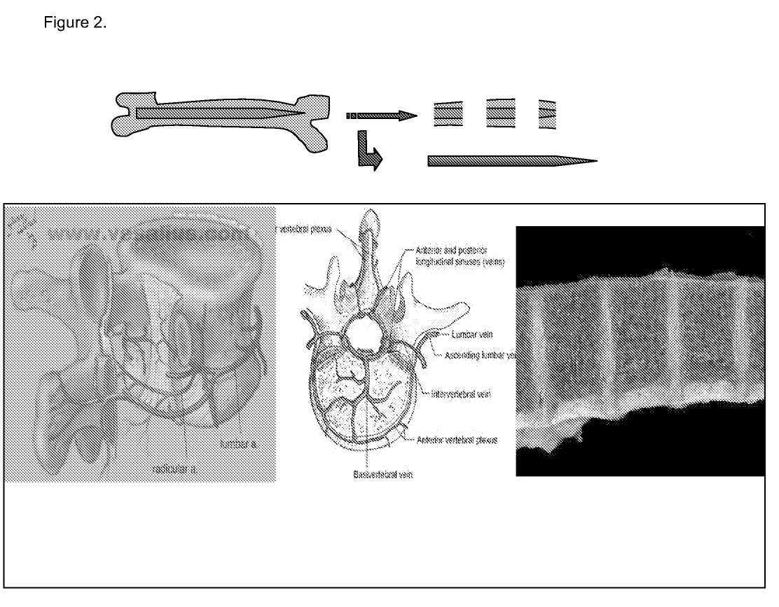

FIG. 3. Preparation steps of individual vertebrae, followed by perfusion with syringe.

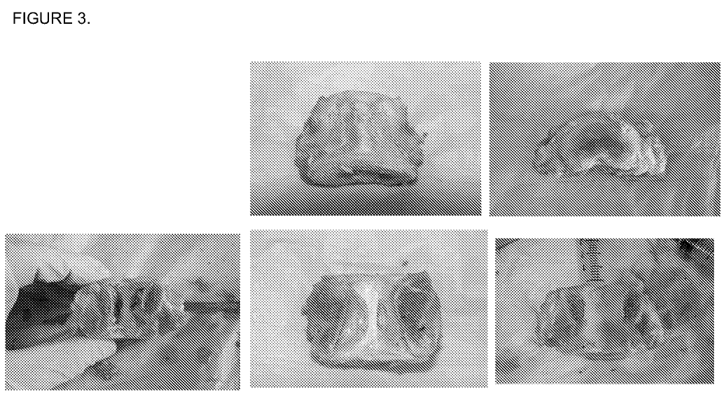

FIG. 4. Porcine Vertebrae (Top) biopsy of vertebrae purfused for 7 days, biopsy site patched with bone wax, and resulting core sample. (Bottom) Syringe pump setup.

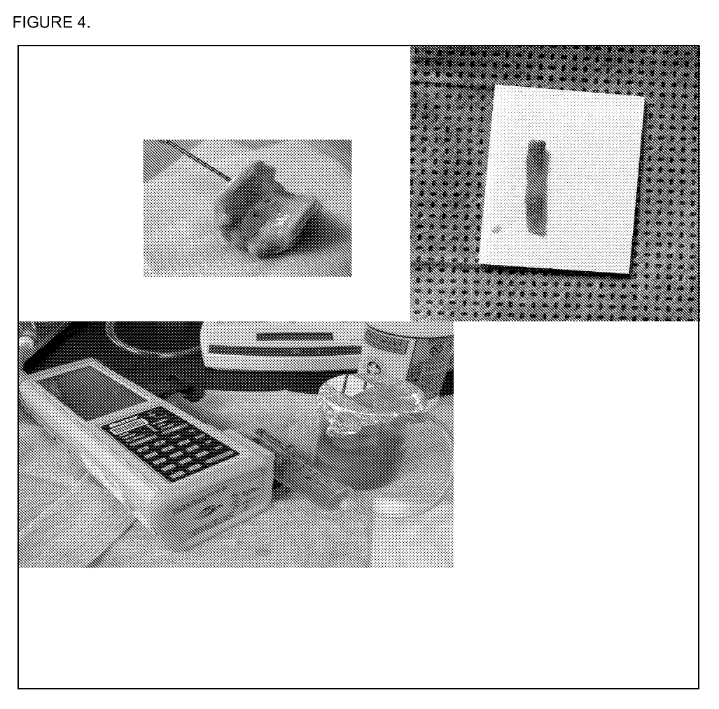

FIG. 5. Example of Integrated Bone Marrow Bioreactor perfusion a single vertebrae: Includes gas supply, oxygenation, pump, media, warming bath and stirrer.

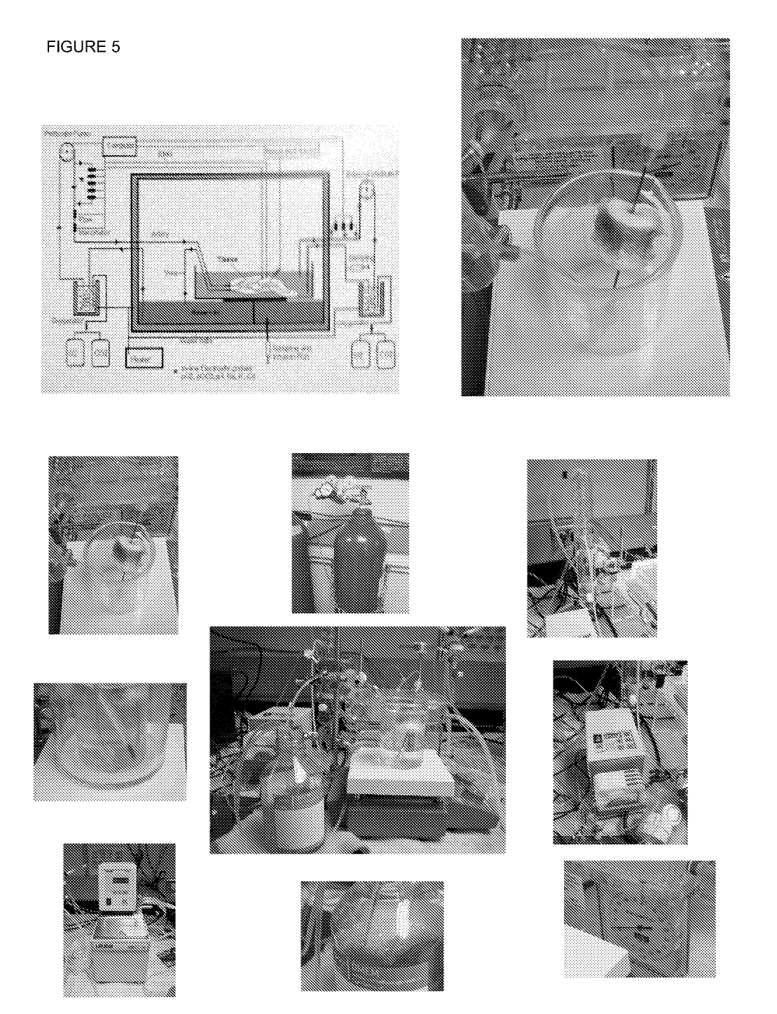

FIG. 6. A. Viability of flushed cells from intact incubated vertebrae, unperfused (red), 0.5 ml/hr of media (green), 1 ml/hr (purple), fridge control (blue). B. Incubated Marrow biopsy cores, maintaining viability of 50% by D+16.

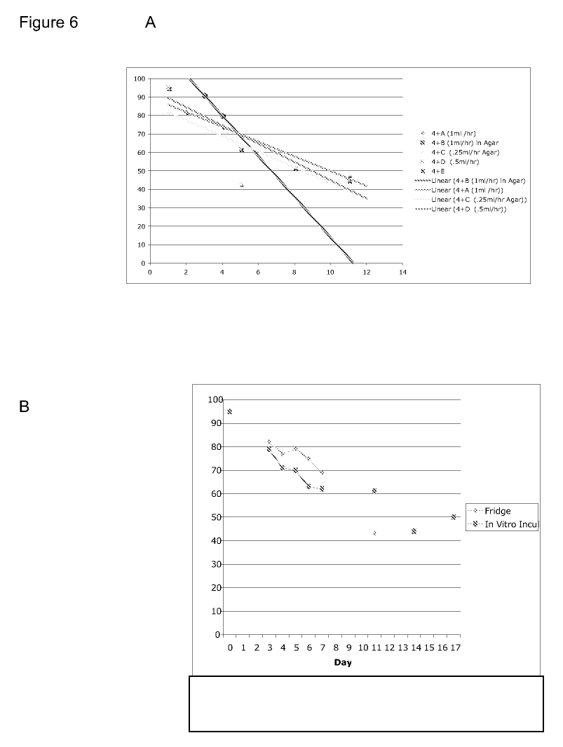

FIG. 7. Vertebral biopsy after 7 days of perfusion (top) from Bioreactor perfused 1.5 ml/hr compared to non-perfused sample (apoptotic, losing structural integrity). Multiple serial biopsies demonstrated intact normal appearing tri-lineage hematopoiesis compared to non-perfused marrow units.

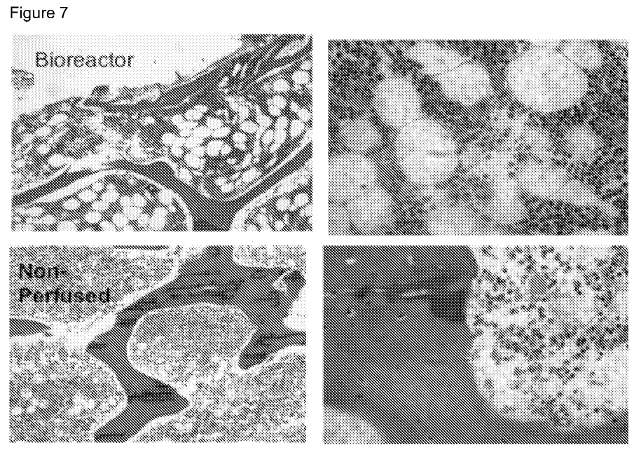

FIG. 8 is a schematic illustrating an exemplary system of the invention.

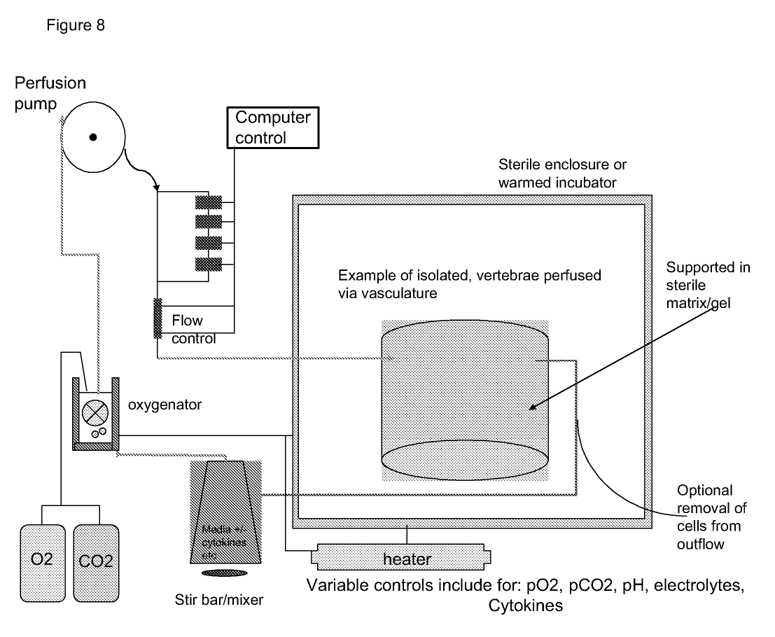

FIG. 9 is a schematic illustrating A) multiple passively perfused BMU channels B) syringe pump via proximal/distal entry site C) cultured core biopsies, D) perfused cores E) directly via vasculature.

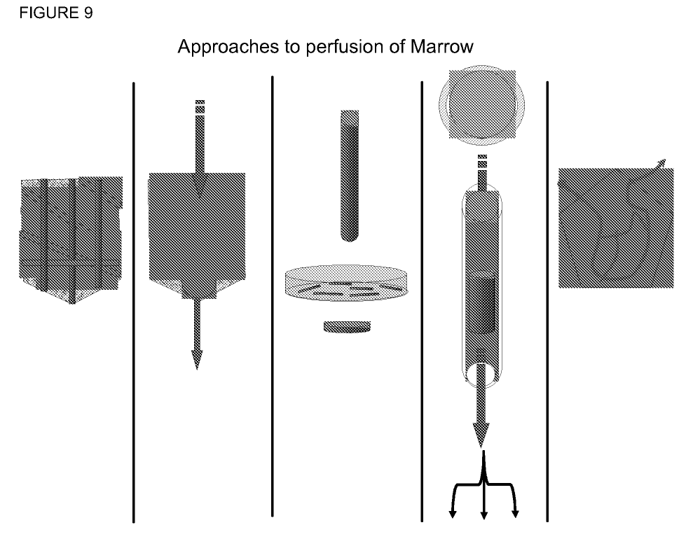

### DETAILED DESCRIPTION OF THE EMBODIMENTS

Devices and methods are provided for the ex vivo maintenance of bone marrow ‘niches’ or normal physiologic home of bone marrow (BM). In the methods of the invention, intact bone marrow, which optionally includes bone normally associated with the marrow, and/or additional protective coatings, is perfused with a medium that supports bone marrow cell viability in an in vitro culture system. Perfusion of the bone marrow is important to the function, and the systems of the invention include such elements that are required to maintain perfusion, which may include a pump, optionally in combination with controllers, filters, media reservoir, etc., and such vessels as are required to provide for perfusion, e.g. tubing, etc. In some embodiments of the invention, the bone marrow is human bone marrow, e.g. a “core” comprising the non-bony cells of the marrow, which are maintained in the original three-dimensional structure found in vivo. In other embodiments the bone marrow includes bone (whole bone, e.g. vertebrae, bone sections, etc.) normally contacting the marrow in vivo.

The systems of the invention allow the study of normal human hematopoiesis, including the location(s) within the normal niche of HSC and progenitor populations, real time study and imaging of homing of HSC via the vasculature, mobilization, and in-situ differentiation. In some embodiments the system provides a tool for ex vivo expansion of hematopoietic stem cells. In addition to the study of normal mechanisms, the systems of the invention provide a means of studying human HSC engraftment in the setting of a variety of conditioning regimens (including radiation, chemotherapy, novel HSC targeted antibody based conditioning). The systems also allow drug screening to determine the effects of drugs on normal or abnormal (i.e. in settings of genetic disease like sickle cell, or malignancies like leukemia) bone marrow and bone marrow niches.

Before the present invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sample” includes a plurality of such samples and reference to “the molecule” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Bone marrow. As used herein, the term bone marrow refers to the cellular tissue found in the hollow interior of mammalian and avian bones, particularly mammalian. Bones of interest for obtaining marrow include long bones, hip bone, breast bone, skull, ribs, vertebrae and shoulder blades, etc. “Mammal” refers to any animal classified as a mammal, including humans, domestic and farm animals, and zoo, sports, or pet animals, such as dogs, cats, cattle, horses, sheep, pigs, goats, rabbits, etc., particularly including humans. Cellular constituents of bone marrow include, without limitation, adipose and pre-adipocytic cells, stromal cells, fibroblastic cells, endothelial cells, e.g. in the blood vessels of the marrow, endothelial stem cells, etc., hematopoietic cells, including hematopoietic stem cells and progenitors, mesenchymal cells, including mesenchymal stem cells, osteoclasts, osteoblasts, chondrocytes, etc.

Intact bone marrow, as used herein refers to the cells and the arrangement of cells in three-dimensional space, substantially as it is found in bone marrow in vivo.

An intact niche refers to the set and arrangement of bone marrow cells that provide an environment in which a stem cell is maintained, and/or in which the stem cell growth and response to exogenous signals is provided for. Niches of interest include hematopoietic niches, mesenchymal niches, etc. Intact niches may include intact bone marrow cores, e.g. a bone marrow biopsy core sample taken from a living human or animal; or may include bony portions, e.g. whole bones such as vertebrae, etc., or fragments or slices of bone. Bones of interest include, without limitation, individual vertebrae (whole or in various parts and sections), femur, humerus, clavicles, sternum, ribs, etc.

In addition to larger whole or bone fragments, e.g. vertebrae, a BMU can consist of bone marrow biopsy type cores (which typically range from about 0.25 cm to about 2 cm in length), and which may be taken with an 8-14 gauge commercially available marrow biopsy needle. These marrow cores maintain their three dimensional structure, and comprise cells undergoing hematopoiesis. Core biopsies maybe obtained from a living donor, e.g. iliac crest biopsy procedure, or from an organ/cadaveric donor. In other embodiments, core biopsies are obtained from a cancer, e.g. leukemia, myeloma, etc. patient.

Preparation of vertebrae may include, without limitation, removal of the spinal column from an organ donor in a sterile fashion. Individual vertebrae are separated using a scalpel or powered cutting instrument. The spinal cord and other nervous tissue is removed, and optionally the posterior elements of the vertebrae are removed to leave the vertebral body. The anterior aspect (with visible vasculature bed) is optionally packed with sterile bone wax or other material to close off any exposed vasculature. Individual vertebrae can be removed from the spinal column, and excess muscle removed or trimmed from the individual vertebrae.

Cells of the bone marrow or cells introduced into the bone marrow environment may be genetically altered in order to introduce genes. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over an 8-16 h period, and then exchanged into growth medium for 1-2 days. Genetically altered cells are selected using a drug selection agent such as puromycin, G418, or blasticidin, and then recultured.

The cells of this invention can also be genetically altered in order to enhance their ability to be involved in tissue regeneration, or to deliver a therapeutic gene to a site of administration. A vector is designed using the known encoding sequence for the desired gene, operatively linked to a promoter that is constitutive, pan-specific, specifically active in a differentiated cell type, etc. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages.

Stem cells may be characterized by both the presence of markers associated with specific epitopes identified by antibodies. Stem cells of interest include hematopoietic stem cells; neural crest stem cells (see Morrison et al. (1999) Cell 96:737-749); mesenchymal stem cells; mesodermal stem cells; etc. The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

Stem cells which are employed may be fresh, frozen, or have been subject to prior culture. They may be fetal, neonate, adult, etc. Hematopoietic stem cells may be obtained from fetal liver, bone marrow, blood, particularly G-CSF or GM-CSF mobilized peripheral blood, or any other conventional source. Cells for engraftment are optionally isolated from other cells, where the manner in which the stem cells are separated from other cells of the hematopoietic or other lineage is not critical to this invention. If desired, a substantially homogeneous population of stem or progenitor cells may be obtained by selective isolation of cells free of markers associated with differentiated cells, while displaying epitopic characteristics associated with the stem cells.

### Methods of Perfusion and Maintenance Ex Vivo

The system of the invention provides for perfusion of bone marrow units (BMU) with medium that provides for ex vivo cell viability. Excluding red blood cells, in the system of the invention at least about 5%, at least about 10%, at least about 20% of more of the initial cell population remains viable for at least about one day, at least about three days, at least about one week, or more. Cells include the supportive and hematopoietic and other stem/progenitor cells contained in the bone marrow unit.

By adjusting the perfusion rate, oxygenation, media and supplements, as discussed below, the perfused BMU can be viably maintained over days to weeks. The BMU may be seeded with host or donor cells, including stem cells, of various populations, and used to screen/test effects of various molecules, malignant cells etc. In some embodiments multiple BMU are obtained from a single donor, in other embodiments BMU from multiple donors are utilized.

Perfusion allows fresh and/or recirculated, oxygenated and nutrient containing media to be circulated in a continuous, non-continuous, or pulsed manner. A pump and controller may be included in the system for this purpose, which pump is operably connected to a conduit through which the perfusate is circulated. The perfusate may be pumped or moved at rates varying from about 0.1 ml per hour up to and including about 50-100 per minute, depending on the specific configuration. A system of the invention may control variables of perfusion and conditions of the bone marrow elements, where the system comprises one or more of the following elements 1) media pump which may have an integrated or separate mechanism for flow control; 2) oxygenator with a supply of O₂; N₂; CO₂, or controllable/definable/adjustable mixtures thereof; 3) sterile housing for the cellular elements. Media exiting the BMU can be discarded, or recycled and reperfused following re-oxygenation and or removal of waste products produced by cells, and cells from the BMU can be removed from the exiting perfusate/media and later utilized in a variety of ways, including for cell isolation (i.e. RBC, WBC, HSC) for various clinical applications.

Perfusate may comprise any suitable medium, including conventional media e.g. DMEM, RPMI, MEM, and the like, including standard or modified cell culture medium, with or without augmentation with serum, including bovine, fetal calf, human, serum replacement, etc., e.g. at a concentration of from about 1% to about 50%. Other factors may be included as known in the art, e.g. glutamine, 2-ME, amino acids, antibiotics, etc., and other known compounds which support various aspects of cell viability, growth and expansion/division. The cellular components of the system are usually maintained in physiologic temperature settings, usually from about 32-37° C. degrees, optionally within a larger incubator, and may be perfused with warmed media, although in some instances cooler temperatures are utilized.

Handling and perfusion of all marrow elements is done in a sterile fashion, e.g. under a conventional tissue culture flow hood, to avoid and decrease risk of contamination. Antibiotics, e.g. penicillin, streptomycin, etc., antifungals, e.g. amphotericin, and the like are optionally included in the perfusate to decrease the likelihood of infection of the system. Bone Marrow Units are optionally immersed briefly or ‘painted’ in betatine solution followed by rinse with saline or media, to eliminate or decrease any surface bacterial load which may be incurred during harvest or during perfusion. Bone marrow units may also be coated with a protective layer, e.g. wax, polymer, etc. as a passive protective layer.

The perfusate may additional act as a vehicle for the introduction of cells, and other biologically active agents, e.g. virus, bacteria, polynucleotides, drugs and drug candidates, cytokines and other biologically active polypeptides, antibodies, etc. Cytokines of interest include GCSF, GM-CSF, TPO, EPO, SCF, IL-2, IL-4, IL-7, and other cytokines, proteins or chemicals in media or in implanted pellets or reservoirs which can stimulate, modify or drive hematopoiesis to a desired pathway, e.g. the use of thrombopoietin (TPO) to stimulate platelet production, the use of erythropoietin (EPO) to stimulate RBC production, and the like.

Methods of perfusion include direct perfusion via the vascular supply, in which oxygen and nutrient containing media is perfused, pumped or otherwise passaged through the existing vascular vascular bed, arterial and or/venous. In such methods a conduit, e.g. small bore tubing, is operably connected to the vasculature of the intact bone marrow.

In one such embodiment, using the example of the single vertebrae, the arterial and venous supply (on one or both sides of the vertebrae) are cannulated, and a small catheter placed into the arterial as well as afferent venous supply. These cannulized vessels are tied off (using suture or other standard microvascular surgical methods) or otherwise sealed, e.g. with bone wax, to enable a ‘closed’ perfusion via the existing vascular supply of the BMU, as shown in the Example.

Alternatively, single or multiple bone marrow units, e.g. cores, vertebrae, etc. are placed in tissue culture dishes, with various supportive media exchanged at a suitable rate, while within a conventional incubator, e.g. with adjustable CO₂, N₂, and O₂. With direct perfusion, a single or multiple catheter or syringe may be placed at the superior and inferior, or lateral aspects of the BMU, e.g. vertebrae. Perfusion by syringe, syringe pump, or other pump is then initiated. The vascular bed may be closed off with a suitable sealant, so that all perfused media moves thru the vertebrae and out the exiting syringe.

In some embodiments, perfusion is enhanced by contacting the bone marrow, e.g. a bone marrow core, with one or a plurality of conduits that perfuse the bone marrow unit with medium. Conduits of interest include, without limitation, flexible or rigid tubing (for example, sterile intravenous fluid line) with a snug/tight or looser fit and may be operably connected to a controller and pump for continuous or pulsed perfusion. Multiple core biopsy marrow specimens may be arranged in series, e.g. two or more BMU within a single ‘tube’; or arranged in parallel, with a pump system driving perfusion to a plurality of BMU, which are each in separate tubes.

Specific embodiments of interest includes perfusion of a single, or multiple (i.e. in series or parallel) human or animal sources vertebrae (which can commonly be removed from donors in a sterile fashion at the time of organ harvesting such as in the case of organ donors).

### Methods of Use

In some embodiments of the invention, core biopsies are obtained from a patient with a hematologic malignancy, e.g. at the time of diagnosis; relapse etc.; and utilizing perfused biopsies from the donor screen various chemotherapeutic modalities for their cytotoxicity against the patients specific malignancy, thus providing a means to choose and optimize (personalize) the patients following treatment regimen. For example multiple biopsy cores may be taken from a newly diagnosed leukemia patient prior to therapy. The core samples are perfused and one or more of the cores receive a separate chemotherapeutic or other cytotoxic treatment. Following administration of the drug(s), the presence of any residual malignant cells can be determined. In such a way screening of novel, or existing drugs or other modalities could be tested on viable malignant cells within the marrow niche as an improved means to test for cytotoxicity or other measures of effectiveness against a particular cancer.

In other embodiments the systems of the invention are utilized in the expansion of endogenous cells or exogenous cells, e.g. hematopoietic stem cells (HSC), and/or progenitor populations (including common myeloid progenitors (CMP), common lymphoid progenitors (CLP), or stem cells and progenitors derived from embryonic stem cells (ESC) or induced pluripotent stem (IPS) stem cell lines. Such cells, when introduced into perfused bone marrow system of the invention can expand, divide and differentiate; which finds use in expansion of the starting cell population, and in derivation of differentiated cells derived there from, such as RBC, WBC, platelets, neutrophils, and the like.

Cells, particularly hematopoietic cells, can be removed from the BMU by various means, including flushing (under normal perfusion or higher perfusion rates), and crushing the BMU (i.e. mortal and pestle type method). Host or donor derived cells may be further isolated/separated and enriched by various methods known in the art, e.g. fluorescent activated cell sorting (FACS), immunomagnetic bead enrichment, plating, and the like.

Cells from the BMU, in mixed, enriched or purified compositions can be utilized in a variety of settings, including transplantation of the cells or platelets for anemia, thrombocytopenia, hematopoietic stem cell transplantation (both autologous or allogeneic transplantation), and the like.

The BMU can be optionally “conditioned” to remove host hematopoietic and other elements, by means including but not limited to irradiation, or chemotherapy/chemicals which are fully or partially myeloablative, meaning they kill a portion or all of the hematopoietic elements within the BMU, and allow for engraftment of exogenous cells (including HSC, progenitors, HSC and IPS derived hematopoietic or other stem/progenitor cells).

An array of BMU/Bone Marrow Bioreactors can be constructed to include a plurality of BMU, e.g. cores, vertebrae, etc., which may be obtained a single or a plurality of donors. The array may be perfused in parallel or serially, to enable a large total mass of marrow element to be maintained, including within an incubator, or maintained by external temperature and gas control. Arrays of BMU optionally comprise a single control element interface to adjust various aspects such as perfusion rates, temperature, CO₂ and O₂ concentrations. In such a manner a single organ donor, whose vertebrae are removed could provide 2 or more vertebral units, each of which is separated out and perfused in parallel, to provide a substantial bioreactor to produce various blood products, or in which to expand donor or host derived stem/progenitors which at various points could be harvested from the system (by biopsy, flushing, crushing or other methods), to include regular removal of cells from the system which flow out of the media perfusate.

Perfused BMU/BMB can be obtained from donors/patients with malignancy (such as various leukemias, lymphomas, or metastatic disease to the bone marrow), or a BMU from a healthy donor could be infused with malignant cells and various drugs or molecules screened for their action upon malignant cells, cancer stem cells etc. For example blasts from a patient with acute lymphoblastic leukemia (ALL) could be placed into a BMU (with or without prior conditioning to possibly enhance engraftment of these cells), and after these malignant cells were living within the BMU, various drugs, radiation or other modalities applied to the BMU, which subsequently could be assayed (by biopsy or other means) to measure the effect of various drugs/compounds on the malignant cells (measure cell death, or other changes, both to healthy and malignant components of the BMU).

### Screening Assays

Perfused BMU/BMB can be utilized to screen and test various compounds, drugs, small molecules, iRNA, or other molecules, as to their effect on various components of the bone marrow and hematopoietic niche, their effect on hematopoiesis, MSC, differentiation, mobilization of HSC and other cells. Of particular interest are screening assays for agents that are active on human cells. A wide variety of assays may be used for this purpose, including determination of cell growth, differentiation and functional activity; production of factors; and the like.

In screening assays for biologically active agents, anti-proliferative drugs, etc. the perfused BMU/BMB composition is contacted with the agent of interest, and the effect of the agent assessed by monitoring output parameters, such as expression of markers, cell viability, and the like. The cells may be freshly isolated, cultured, genetically altered, and the like. The cells may be environmentally induced variants of clonal cultures: e.g. split into independent cultures and grown under distinct conditions, for example with or without drugs; in the presence or absence of cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:l to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluorescence, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### Experimental

Studies with murine bone marrow and with human size porcine vertebrae have demonstrated the ability to maintain the endogenous marrow niche in vitro and the biologically active HSC it contains.

Murine Model of Ex-Vivo Niche: Briefly, femur and tibia from 6-12 week old BA or BA-GFP mice were harvested, and sectioned into ⅓^{rd} bone marrow fragments (BMF) with proximal and distal ends removed to facilitate perfusion of the marrow cavity. In addition, whole marrow ‘plugs’ were flushed from tibia. These were then cultured in normal or low oxygen, in X-vivo media with 15% FCS with or without supplemental cytokines (Flt2, SCF, TPO). At various time points bone fragments and intact marrow plugs were removed, cells flushed, stained with antibodies to HSC markers, and analyzed by flow cytometry, CFU assay, as well as also transplanted into sublethally irradiated (500 Rad) Ly5.2 recipients.

### Results

BMF contained 35-72% viable cells as measured by trypan blue exclusion at up to 14 days. In 4 to 11 day old BMF, a population of cells with long-term HSC phenotype of Slam+, c-Kit, Sca-1 Positive, lineage negative cells (KLS) cells were detectable. In BMF transplanted recipients, donor derived cells engrafted and were detectable >4 months post transplant. Mean BMF donor derived granulocyte engraftment was 5.2 to 22.1%, compared to mean 19% from fresh, control BM cells, suggesting long-term engraftment was derived from HSC maintained within cultured BMF. Low oxygen culture as well as cytokines enhanced but was not required for BMF donor derived cell engraftment.

Large animal model: Fresh thoracic and lumbar vertebrae from large juvenile swine were harvested and separated in a sterile fashion. then maintained in culture as: A) non-perfused in media, B) syringe perfused (Baxter AS50 syringe pump) from 0.5 to 1.5 ml/hr of RPMI+FCS media, or C) by surgically cannulated vertebral artery and vein and maintained on a bioreactor. The bioreactor enables control of infusion rate, temperature, oxygenation.

Vertebrae underwent core biopsies every 1-2 days and cells from biopsy cores and flushed whole vertebrae were analyzed for viability and by histology. Additionally multiple core biopsies were obtained from fresh vertebral specimens, and cultured in 6 well plates, or continuously perfused within 6 guage sterile tubing (0.1-0.5 ml/hour media).

In porcine vertebral bodies, non-perfused vertebrae had <10% viability by day 4 compared to 20% viability if perfused by pump at 0.5 ml/hr or a mean of 49% viability when perfused at >1 ml/hr. Histology revealed maintained marrow structure and healthy appearing cells exhibiting tri-lineage hematopoiesis in marrow cores from perfused vertebrae in contrast to unperfused marrow samples.

### CONCLUSIONS

These results demonstrate that the intact HSC niche can be maintained in vitro and further optimization of this approach may provide a novel means to study murine and eventually human HSC and niche in vitro.

What is claimed is:

1. A system for ex vivo maintenance of bone marrow biological function, comprising:
a bone marrow unit comprising intact bone marrow, which bone marrow is perfused with medium that provides for viability of cells within the bone marrow for a period of at least one day.

2. The system of claim 1, wherein the bone marrow unit is a bone marrow core.

3. The system of claim 2, wherein the bone marrow core is sealed with a protective coating.

4. The system of claim 1, wherein the bone marrow unit comprises bone normally contacting the marrow in vivo.

5. The system of claim 4, wherein the bone marrow unit is a vertebrae.

6. The method of claim 4, wherein the bone marrow unit is a long bone or section thereof.

7. The system of claim 1, wherein the bone marrow unit is human.

8. The system of claim 1, wherein the bone marrow unit is a non-human mammal.

9. The system of claim 1, wherein the biological function is hematopoiesis.

10. The system of claim 1, wherein the bone marrow unit is operable connected to at least one conduit for perfusion.

11. The system of claim 10, wherein the at least one conduit is operably connected to a pump.

12. The system of claim 11, wherein the pump is operably connected to a controller.

13. The system of claim 12, wherein the at least one conduit is operably connected to the vasculature of the bone marrow unit.

14. The system of claim 12, wherein the at least one conduit is operably connected to a non-vascular element of the bone marrow unit.

15. The system of claim 12, comprising a plurality of bone marrow units.

16. The system of claim 15, wherein the plurality of bone marrow units are arranged in series relative to the conduit.

17. The system of claim 15, wherein the plurality of bone marrow units are arrange in parallel relative to the conduit.

18. The system of claim 15, further comprising cells exogenous to the bone marrow unit.

19. The system of claim 18, wherein the exogenous cells are hematopoietic stem or progenitor cells.

20. The system of claim 1, wherein the medium comprises biologically active agents.

21. The system of claim 20, wherein the biologically active agents are cytokines.

22. A method for the maintenance of bone marrow biological function ex vivo, the method comprising:
maintaining a bone marrow unit in a system set forth in claim 1.

23. The method of claim 22, wherein cells are removed from the system.

24. The method of claim 23, wherein the cells are hematopoietic cells.

25. The method of claim 22, wherein hematopoietic cells are expanded in number in the system.

26. The method of claim 25, wherein hematopoietic stem or progenitor cells are added to the system for expansion.

27. A method for screening candidate agents for an effect on bone marrow, the method comprising:
contacting a candidate agent with a system set forth in claim 1; and
determining the effect of the agent on bone marrow biological activity.
